(19) United States
(12) Patent Application Publication
(10) Pub. No.: US 2023/0147467 A1
(43) Pub. Date: May 11, 2023

(54) Novel Contrast Agents and Imaging Methods for Early Detection of Diseases

(71) Applicant: Ishtar Faisal Algburi, London (CA)

(72) Inventor: Ishtar Faisal Algburi, London (CA)

(21) Appl. No.: 17/523,911

(22) Filed: Nov. 11, 2021

(51) Int. Cl. A61K 49/04

(52) U.S. Cl. CPC A61K 49/0438

(57) ABSTRACT

This invention relates to novel contrast agents for detection of diseases by using any or both or all of CT, SPECT and PET scans. Previously, there was no way to use these novel contrast agents for imaging and early detection of neurodegenerative diseases, multiple sclerosis disease, concussion, and traumatic brain injury. Also, previously, there was no way to use Computed Tomography (CT) imaging to early detect and map the neurodegenerative diseases. Embodiments of the present invention use a novel contrast agent is disclosed for early detection of diseases by using any or both or all of CT, SPECT and PET. Also, these novel contrast agents can be used for detection of amyloid beta, tau protein, alpha-synuclein protein, and aggregation proteins in neurodegenerative diseases and inflammation in many diseases such as neurodegenerative diseases, cancer and other inflammatory diseases, blood brain barrier leakage or break down, aggressive cancer tissue, brain activation in neurodegenerative diseases and other diseases, and hypoxia in tissue. Also, these novel contrast agents increase there binding in diseases compared to healthy subjects, therefore induce higher contrast in diseases such as in neurodegenerative diseases, cancer and other inflammatory diseases, blood brain barrier leakage or break down, aggressive cancer tissue, brain activation in neurodegenerative diseases and other diseases, and hypoxic tissue.

### CROSS REFERENCE TO RELATED APPLICATION

This application claims the benefit of priority to U.S. Provisional Pat. Application No. 63/118,723, filed on Nov. 26, 2020, the entirety of which is incorporated by reference for any and all purposes.

### BACKGROUND

Embodiments of the invention described in this specification generally relate to disease detection by imaging, and more particularly, to novel contrast agents and imaging methods for early detection of diseases by using novel contrast agents for detection of diseases by using any or both or all of computed tomography (CT), single-photon emission computed tomography (SPECT) and positron emission tomography (PET) scans.

Positron emission tomography (PET) scanners are used for detection and mapping amyloid beta plaques, and neurofibrillary tangles in the brains of living people. PET scanners use radiotracers (“tracers”) and/or contrast agents to detect disease. When injected into the bloodstream of a patient, tracers or contrast agents cross quickly into the brain, where they bind to amyloid plaques or neurofibrillary tangles to mark them with emissions of mild radioactivity. Amyloid beta imaging is highly useful to enable people to begin therapy early enough in time to avoid or to significantly delay the development of neurodegenerative diseases. Amyloid beta proteins are aggregated and accumulated at the extracellular space and form large accumulations of aggregation proteins, called amyloid beta plaques, which cause neuronal death. In the progression of the disease, amyloid beta plaques precede tau tangles, and both cause eventual neural loss, the accumulation of amyloid in the brain has been identified as an early biomarker of some diseases, such as Alzheimer’s disease. There is no current method used for early detection of neurodegenerative diseases before 10-20 years of the symptoms appear, by using these novel contrast agents in any or both or all CT, SPECT and PET scans, it is possible to detect these diseases early before the symptoms appear.

Method of imaging and detection the amyloid beta, tau protein, alpha-synuclein protein, and aggregation proteins in neurodegenerative diseases and inflammation in many diseases, aggressive cancer tissue, brain activation in neurodegenerative diseases and other diseases by administration of the contrast agents like: (i) labelling the compounds listed below by iodine (I) or bromine (Br) or any other elements used in CT contrast agent with same property of iodine and bromine as a contrast agent for CT scan to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases. (ii) labelling the compounds listed below by radiotracers such as iodine-123, thallium-20, technetium-99m, xenon-133, and fluorine-18 and other radiotracers which emitted gamma rays detected by SPECT to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases. (iii) labelling the compounds listed below by radiotracers or isotopes such as iodine-124, iodine-131, fluorine-18, carbon-11, nitrogen-13 and other radiotracers or isotopes that used in PET scan and which emitted positrons detected by PET to detect amyloid beta, tau protein, alpha-synuclein protein, and aggregation proteins in neurodegenerative diseases, inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases. These compound to be labelled in (i), (ii), (iii) such as:

{Polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; compounds like bromophenols and their derivatives, iodophenols and their derivatives, bromoquinols and their derivatives, iodoquinols and their derivatives which contain Br or I can be used without or with labeling from method mentioned in (i).

Blood brain barrier (BBB) leakage or breakdown early in many diseases such as cancer, AD, PD, HD, ALS, MS, HIV-1-associated dementia and other diseases. Also, BBB is identified as potential early warning sign for neurodegenerative diseases and associated with inflammatory and immune responses and there are potential connections between blood brain barrier dysfunction and several neurodegenerative diseases, including Alzheimer’s and Parkinson’s diseases, HD, ALS, MS and other disease. Increase permeable blood brain barrier may allow more toxic amyloid and tau proteins to enter the brain. BBB breakdown can be used to early detection of the diseases. In most cases, current method now to detect BBB leakage or breakdown by using MRI contrast agent, such as Gadolinium, to better identify the blood brain barrier leakage. However, Gadolinium is expensive and has many side effects on humans, especially to humans with kidney diseases.

There is also evidence showing that activation of glial cells, including microglia and astrocytes, plays an important role in the inflammatory signalling in the diseases such as neurodegeneration diseases. Also, activation of microglia by amyloid beta, tau protein, and APP also results in an upregulation of inflammation. Method of imaging brain activation and used this method for early detection of the diseases in AD, PD, HD, ALS, MS, cancer and all other neurodegenerative disease. Method of imaging inflammation in AD, MS, PD, HD, ALS, cancer and other neurodegenerative diseases by administration and labelling the compounds listed above by ways from (i) to (iii) by CT, SPECT and PET machine respectively.

Therefore, what is needed is a way to use novel contrast agents in any or both or all of CT, SPECT and PET to detect amyloid beta, tau protein, alpha-synuclein protein, and aggregation proteins in neurodegenerative diseases and inflammation in many diseases such as neurodegenerative diseases, cancer and other inflammatory diseases, blood brain barrier leakage or break down, aggressive cancer tissue, brain activation in neurodegenerative diseases and other diseases, and hypoxia in tissue by binding and accumulation of the these labeled novel contrast agents to these proteins and diseases.

### BRIEF DESCRIPTION

The method presented in this invention is using contrast agents for detection of diseases by using any or both or all of computed tomography (CT), single-photon emission computed tomography (SPECT) and positron emission tomography (PET) scans, these contrast agents can detect amyloid beta, tau protein, alpha-synuclein protein, and aggregation proteins in neurodegenerative diseases and inflammation in many diseases such as neurodegenerative diseases, cancer and other inflammatory diseases, blood brain barrier leakage or break down, aggressive cancer tissue, brain activation in neurodegenerative diseases and other diseases, and hypoxia in tissue by binding and accumulation of the contrast agents to these proteins and diseases. Method of imaging and detection the amyloid beta, tau protein, alpha-synuclein protein, and aggregation proteins in neurodegenerative diseases and inflammation in many diseases, aggressive cancer tissue, brain activation in neurodegenerative diseases and other diseases by administration of the contrast agents like:

(i) labelling the compounds { polyphenols such as curcumin, rosmarinic acid, phenylindane, silibinin, silymarin, thearubigins, stilbene derivatives, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; (compounds like bromophenols and their derivatives, iodophenols and their derivatives, bromoquinols and their derivatives, iodoquinols and their derivatives which contain Br or I can be used without or with labeling)}

by iodine (I) or bromine (Br) or any other elements used in CT contrast agent with same property of iodine and bromine as a contrast agent for CT scan to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases.

(ii) labelling the compounds { polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, stilbene derivatives and others, except stilbene derivatives for amyloid beta detection only by SPECT; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; compounds like bromophenols and their derivatives, iodophenols and their derivatives, bromoquinols and their derivatives, iodoquinols and their derivatives} by radiotracers such as iodine-123, thallium-20, technetium-99m, xenon-133, and fluorine-18 and other radiotracers which emitted gamma rays detected by SPECT to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases.

(iii) labelling the compounds { polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, stilbene derivatives, and others, except stilbene derivatives for amyloid beta detection only by PET; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; compounds like bromophenols and their derivatives, iodophenols and their derivatives, bromoquinols and their derivatives, iodoquinols and their derivatives} by radiotracers or isotopes such as iodine-124, iodine-131, fluorine-18, carbon-11, nitrogen-13 and other radiotracers or isotopes that used in PET scan and which emitted positrons detected by PET to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases.

These labeled contrast agents have binding ability to human proteins such as binding to human serum albumin (HSA) and/or binding to inflammatory tissue which can be used these compounds as contrast agents to detect amyloid beta, tau protein, alpha-synuclein protein, and aggregation proteins in neurodegenerative diseases and inflammation in many diseases such as neurodegenerative diseases, cancer, and other inflammatory diseases, aggressive cancer tissue, brain activation in neurodegenerative diseases and other diseases. The protocol of using these contrast agents is similar to the CT, SPECT, and PET protocols of using contrast agents in regular CT, SPECT, and PET scans for detection the other diseases such as could be similarity in doses and scan time for CT, SPECT, and PET. For example, CT, SPECT, and PET contrast agents such as labelling any or both or all of epigallocatechin gallate, quercetin, rosmarinic acid, and/or any other compounds mentioned above in the description with:

(i): iodine (I) or bromine (Br) or any other elements used in CT contrast agent with same property of iodine and/or bromine as a contrast agent for CT scan to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases.

(ii) radiotracers such as iodine-123, thallium-20, technetium-99m, xenon-133, and fluorine-18 and other radiotracers which emitted gamma rays detected by SPECT to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases.

(iii) radiotracers or isotopes such as iodine-124, iodine-131, fluorine-18, carbon-11, nitrogen-13 and other radiotracers or isotopes that used in PET scan and which emitted positrons detected by PET to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of these novel contrast agents to these proteins and diseases.

Which can be binding to human protein such as human serum albumin and/or binding to inflammatory tissue also, can bind to amyloid beta, tau protein, alpha-synuclein protein, and aggregation proteins also, can be accumulated in the inflammatory tissue by multiple binding to the proteins, such as in Parkinson’s disease (PD), epilepsy, ischemic stroke, and Alzheimer’s disease (AD), Multiple sclerosis (MS), Huntington’s disease (HD), Amyotrophic Lateral Sclerosis (ALS), cancer, concussion, traumatic brain injury, and other neurodegenerative diseases and other inflammatory diseases.

Imaging of blood brain barrier (BBB) leakage or break down by labelling agents as described above (i) for CT, (ii) for SEPCT, (iii) for PET of compounds such as (morphine, ascorbic acid (vitamin C), carbidopa, dopamine, betaine, congo red, nipecotic acid, guvacine and other compounds and large polyphenols which can not cross BBB easily). Which cannot cross normal BBB or agent cross normal BBB very slowly, these compounds induce contrast can be detected by CT and/or SPECT or and/or PET for imaging and detection BBB leakage or break down. Also, this detection can be used for early detection of disease which are BBB leakage is hallmark of these diseases such as meningitis, brain abscess, epilepsy, multiple sclerosis, neuromyelitis optica (NMO), de vivo disease, Alzheimer’s disease, HIV encephalitis, systemic inflammation, brain cancer. Compounds cannot cross normal BBB are: morphine, ascorbic acid (vitamin C), carbidopa, dopamine, betaine, congo red and other drugs, which can be used to detect BBB leakage or breakdown at time limit of imaging, such as after 15 mins, 30 mins, 60 mins, or more of administration of the drugs also, compounds that slowly cross BBB or cannot cross BBB readily, can be used to detect BBB leakage or breakdown at time limit of imaging, such as after 30 mins, 60 mins, or more of administration of the drugs such nipecotic acid, guvacine, and other. The CT images contrast and/or SPECT images and/or PET images (brightness of images) increase as these agents cross blood brain barrier (BBB) or as the BBB is leakage or break down.

Imaging of hypoxia in tissue by labelling the compounds in the method described above in (i) for CT, (ii) for SEPCT, (iii) for PET such as compounds having the nitro group, —NO₂, attached to carbon, these agent like Metronidazole, Tolcapone, Nitroxoline, Chloramphenicol, Oxamniquine, Secnidazole and other drugs which contain NO₂ and have ability to donate O₂. The CT images contrast and/or SPECT images and/or PET images (brightness of images) increase as the binding of these agents to the hypoxic tissue increase.

1. The novel contrast agents and imaging methods for early detection of diseases comprising: labelling the compounds { polyphenols such as curcumin, rosmarinic acid, phenylindane, silibinin, silymarin, thearubigins, stilbene derivatives, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, and others; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; (compounds like bromophenols and their derivatives, iodophenols and their derivatives, bromoquinols and their derivatives, iodoquinols and their derivatives which contain Br or I can be used with or without labeling for CT detection)} by iodine (I) or bromine (Br) or any other elements used in CT contrast agent with same property of iodine and/or bromine as a contrast agent for CT scan to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the CT machine; the CT images contrast (brightness of images) increase as the binding of these agents to the target diseases are increased.

2. The novel contrast agents and imaging methods for early detection of diseases comprising: labelling the compounds { polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, stilbene derivatives and others, except stilbene derivatives for amyloid beta detection only by SPECT; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; compounds like bromophenols and their derivatives, iodophenols and their derivatives, bromoquinols and their derivatives, iodoquinols and their derivatives} by radiotracers such as iodine-123, thallium-20, technetium-99m, xenon-133, and fluorine-18 and other radiotracers which emitted gamma rays detected by SPECT to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the SPECT machine; the SPECT images contrast (brightness of images) increase as the binding of these agents to the target diseases are increased.

3. The novel contrast agents and imaging methods for early detection of diseases comprising: labelling the compounds { polyphenols such as curcumin, rosmarinic acid, phenylindane, silibinin, silymarin, thearubigins, theaflavin and its derivatives, theaflavin-3-gallate, tannic acid, catechin, epicatechin, gallocatechin, catechin gallate, gallocatechin gallate, epicatechin gallate, epigallocatechin, and epigallocatechin gallate, stilbene derivatives, and others, except stilbene derivatives for amyloid beta detection only by PET; flavonoids such as luteolin, quercetin, rutin, taxifolin, resveratrol, myricetin, rhein, and others; congo red and it’s analogs such as chrysamine-g; nordihydroguaiaretic acid; cannabinoids such as tetrahydrocannabinol (THC), cannabidiol (CBD), cannabigerol (CBG), and other cannabinoids; phenolic acids such as caffeic acid, ferulic acid, gallic acid, salicylic acid, and other phenolic acid; nonsteroidal anti-inflammatory drugs (NSAIDs) drugs like sodium salicylate, aspirin, diclofenac, celecoxib and other drugs; tannins from brown algae such as phlorotannins include eckol, and it’s derivatives and most polyphenols of seaweed and marine algae; compounds like bromophenols and their derivatives, iodophenols and their derivatives, bromoquinols and their derivatives, iodoquinols and their derivatives} by radiotracers or isotopes such as iodine-124, iodine-131, fluorine-18, carbon-11, nitrogen-13 and other radiotracers or isotopes that used in PET scan and which emitted positrons detected by PET to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the PET machine; the PET images contrast (brightness of images) increase as the binding of these agents to the target diseases are increased.

4. The novel contrast agents and imaging methods for early detection of diseases of claim 1, wherein detecting disease and mapping disease severity comprises using CT machine with the novel contrast agents are mentioned in this invention and PET machine with the novel contrast agents are mentioned in this invention together, and/or using CT machine with the novel contrast agents are mentioned in this invention and SPECT machine with the novel contrast agents are mentioned in this invention together.

5. The novel contrast agents and imaging methods for early detection of diseases of claim 2, wherein detecting disease and mapping disease severity comprises using SPECT machine with the novel contrast agents are mentioned in this invention and CT machine in this invention with the novel contrast agents are mentioned together, and/or using SPECT machine with the novel contrast agents are mentioned in this invention and PET machine with the novel contrast agents are mentioned in this invention together.

6. The novel contrast agents and imaging methods for early detection of diseases of claim 1, comprising: detection of amyloid beta by using the novel contrast agents mentioned in (i) with CT and/or using the novel contrast agent in (ii) with SPECT and/or using the novel contrast agent mentioned in this invention in (iii) with PET to detect amyloid beta in neurodegenerative diseases and other diseases by binding and accumulation of these contrast agent to the target proteins of amyloid beta by way of the CT machine and/or SPECT machine, and/or PET machine; the CT images contrast and/or SPECT images contrast, and/or PET images contrast (brightness of images) increase as the binding of these agents to the target amyloid beta neurodegenerative diseases and other diseases increase.

7. The novel contrast agents and imaging methods for early detection of diseases claim 1, comprising: detection of tau protein by using the novel contrast agents mentioned in this invention in (i) with CT and/or using the novel contrast agent mentioned in this invention in (ii) with SPECT and/or using the novel contrast agent mentioned in this invention in (iii) with PET to detect tau protein in neurodegenerative diseases and other diseases by binding and accumulation of these contrast agent to the target proteins of tau protein by way of the CT machine and/or SPECT machine, and/or PET machine; the CT images contrast and/or SPECT images contrast, and/or PET images contrast (brightness of images) increase as the binding of these agents to the target tau protein neurodegenerative diseases and other diseases increase.

8. The novel contrast agents and imaging methods for early detection of diseases claim 1, comprising: detection of alpha-synuclein protein by using the novel contrast agents mentioned in this invention in (i) with CT and/or using the novel contrast agent mentioned in this invention in (ii) with SPECT and/or using the novel contrast agent mentioned in this invention in (iii) with PET to detect alpha-synuclein protein in neurodegenerative diseases and other diseases by binding and accumulation of these contrast agent to the target proteins of alpha-synuclein protein by way of the CT machine and/or SPECT machine, and/or PET machine; the CT images contrast and/or SPECT images contrast, and/or PET images contrast (brightness of images) increase as the binding of these agents to the target alpha-synuclein protein neurodegenerative diseases and other diseases increase.

9. The novel contrast agents and imaging methods for early detection of diseases claim 2, comprising: detection of aggregation proteins by using the novel contrast agents mentioned in this invention in (i) with CT and/or using the novel contrast agent mentioned in this invention in (ii) with SPECT and/or using the novel contrast agent mentioned in this invention in (iii) with PET to detect aggregation proteins in neurodegenerative diseases and other diseases by binding and accumulation of these contrast agent to the target proteins of aggregation proteins by way of the CT machine and/or SPECT machine, and/or PET machine; the CT images contrast and/or SPECT images contrast, and/or PET images contrast (brightness of images) increase as the binding of these agents to the target aggregation proteins neurodegenerative diseases and other diseases increase.

10. The novel contrast agents and imaging methods for early detection of diseases claim 1, comprising: detection of inflammation in tissue by using the novel contrast agents mentioned in this invention in (i) with CT and/or using the novel contrast agent mentioned in this invention in (ii)with SPECT and/or using the novel contrast agent mentioned in this invention in (iii)with PET to detect inflammation in tissue in neurodegenerative diseases and other diseases by binding and accumulation of these contrast agent to the target proteins of inflammation in tissue by way of the CT machine and/or SPECT machine, and/or PET machine; the CT images contrast and/or SPECT images contrast, and/or PET images contrast (brightness of images) increase as the binding of these agents to the target inflammation in tissue in the inflammatory diseases such as diseases of the skin, diseases of the nervous system, immune diseases, diseases of the musculoskeletal system, concussion, traumatic brain injury, ischemic stroke, cancer, Alzheimer’s disease, Parkinson disease, Huntington’s disease, epilepsy, Down syndrome, Amyotrophic Lateral Sclerosis, Multiple sclerosis and other neurodegenerative diseases and other inflammatory diseases.

11. The novel contrast agents and imaging methods for early detection of diseases claim 2, comprising: detection of brain activation by using the novel contrast agents mentioned in this invention in (i) with CT and/or using the novel contrast agent mentioned in this invention in (ii) with SPECT and/or using the novel contrast agent mentioned in this invention in (iii) with PET to detect brain activation in neurodegenerative diseases and other diseases by binding and accumulation of these contrast agent to the target proteins of brain activation by way of the CT machine and/or SPECT machine, and/or PET machine; the CT images contrast and/or SPECT images contrast and/or PET images contrast (brightness of images) increase as the binding of these agents to the target brain activation neurodegenerative diseases, cancer and other diseases increase.

12. The novel contrast agents and imaging methods for early detection of diseases claim 1, comprising: detection of aggressive cancer by using the novel contrast agents mentioned in this invention in (i) with CT and/or in (ii) with SPECT, and/or in (iii) with PET to detect aggressive cancer tissue by binding and accumulation of these contrast agent to the target proteins of aggressive cancer proteins by way of the CT machine and/or SPECT machine and/or PET machine; the CT images contrast and/or SPECT images contrast, and/or PET images contrast (brightness of images) increase as the binding of these agents to the target aggressive cancer tissue increase.

13. The novel contrast agents and imaging methods for early detection of diseases claim 1, comprising: detection of blood brain barrier leakage or break down by using the novel contrast agents such as labeling agent like (morphine, ascorbic acid (vitamin C), carbidopa, dopamine, betaine, congo red, nipecotic acid, guvacine and large polyphenols, and other compounds which can not cross BBB easily) by labelling these agents with the methods as described above in (i) for CT, (ii) for SEPCT, and (iii) for PET to detect blood brain barrier leakage or break down in neurodegenerative diseases, cancer and other diseases by crossing these agents the blood brain barrier at time limit of imaging, such as after 15 mins, 30 mins, 60 mins, or more of administration of the labelled agents by way of the CT machine and/or SPECT machine, and/or PET machine; the CT images contrast and/or SPECT images contrast, and/or PET images contrast (brightness of images) increase as these agents cross blood brain barrier in neurodegenerative diseases and other diseases increase.

14. The novel contrast agents and imaging methods for early detection of diseases claim 2, comprising: detection of hypoxia by using the novel contrast agents with labelling as mentioned in this invention such as labeling agent like Metronidazole, Tolcapone, Nitroxoline, Chloramphenicol, Oxamniquine, Secnidazole and other drugs which contain NO2 and have ability to donate O2, by labelling the compounds as described above in (i) for CT, (ii) for SEPCT, (iii) for PET to detect hypoxia in tissue by binding and accumulation of these labeled contrast agent to the target proteins of hypoxia by way of the CT machine and/or SPECT machine and/or PET machine; the CT images contrast and/or SPECT images contrast, and/or PET images contrast (brightness of images) increase as the binding of these agents to the target hypoxia in tissue increase.

15. The novel contrast agents and imaging methods for early detection of diseases of claim 1, comprising: administration of CT contrast agents bind to amyloid beta and/or tau protein orally or by injection or administration multiple agents target and bind to amyloid beta and/or tau protein separately to enhance the detection of amyloid beta and/or tau protein in the same time which can be detected by CT machine.

16. The novel contrast agents and imaging methods for early detection of diseases of claim 1, comprising: administration of CT contrast agents bind to synuclein protein and/or proteins aggregation orally or by injection or administration multiple agents target and bind to alpha-synuclein protein and/or proteins aggregation separately to enhance the detection of alpha-synuclein protein and/or proteins aggregation in the same time which can be detected by CT machine.

17. The novel contrast agents and imaging methods for early detection of diseases of claim 1, comprising: administration of CT contrast agents bind to amyloid beta and/or proteins aggregation orally or by injection or administration multiple agents target and bind to amyloid beta and/or proteins aggregation separately to enhance the detection of amyloid beta and/or proteins aggregation in the same time which can be detected by CT machine.

18. The novel contrast agents and imaging methods for early detection of diseases of claim 1, comprising: administration of CT contrast agents bind to amyloid beta and/or inflammation in tissue orally or by injection or administration multiple agents target and bind to amyloid beta and/or inflammation in tissue separately to enhance the detection of amyloid beta and/or inflammation in tissue in the same time which can be detected by CT machine.

19. The novel contrast agents and imaging methods for early detection of diseases of claim 2, comprising: administration of SPECT and/or PET contrast agents bind to amyloid beta and/or tau protein orally or by injection or administration multiple agents target and bind to amyloid beta and/or tau protein separately to enhance the detection of amyloid beta and/or tau protein at the same time which can be detected by SPECT machine and/or PET machine.

20. The novel contrast agents and imaging methods for early detection of diseases of claim 2, comprising: administration of SPECT and/or PET contrast agents bind to synuclein protein and/or proteins aggregation orally or by injection or administration multiple agents target and bind to alpha-synuclein protein and/or proteins aggregation separately to enhance the detection of alpha-synuclein protein and/or proteins aggregation in the same time which can be detected by SPECT and/or PET machine.

21. The novel contrast agents and imaging methods for early detection of diseases of claim 2, comprising: administration of SPECT and/or PET contrast agents bind to amyloid beta and/or proteins aggregation orally or by injection or administration multiple agents target and bind to amyloid beta and/or proteins aggregation separately to enhance the detection of amyloid beta and/or proteins aggregation at the same time which can be detected by SPECT machine and/or PET machine.

22. The novel contrast agents and imaging methods for early detection of diseases of claim 2, comprising: administration of SPECT and/or PET contrast agents bind to amyloid beta and/or inflammation in tissue orally or by injection or administration multiple agents target and bind to amyloid beta and/or inflammation in tissue separately to enhance the detection of amyloid beta and/or inflammation in tissue at the same time which can be detected by SPECT machine and/or PET machine.

23. The novel contrast agents and imaging methods for early detection of diseases of claim 1, comprising: administration of compounds contain at least phenol group and have ability to bind to human protein such as human serum albumin (HAS) or could be as anti inflammatory agents or could be both to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the PET machine and/or SPECT machine, and/or CT machine; the images contrast (brightness of images) increase as the binding of these agents to the target diseases are increased.

24. The novel contrast agents and imaging methods for early detection of diseases of claim 1, comprising: administration of compounds contain at least phenyl group and have ability to bind to human protein such as human serum albumin (HAS) or could be as anti inflammatory agents or could be both to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the PET machine and/or CT machine, and/or SPECT machine; the images contrast (brightness of images) increase as the binding of these agents to the target diseases are increased.

25. The novel contrast agents and imaging methods for early detection of diseases of claim 2, comprising: administration of compounds contain at least phenol group and have ability to bind to human protein such as human serum albumin (HAS) or could be as anti inflammatory agents or could be both to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the PET machine and/or SPECT machine, and/or CT machine; the images contrast (brightness of images) increase as the binding of these agents to the target diseases are increased.

26. The novel contrast agents and imaging methods for early detection of diseases of claim 3, comprising: administration of compounds contain at least phenyl group and have ability to bind to human protein such as human serum albumin (HAS) or could be as anti inflammatory agents or could be both to detect amyloid beta, tau protein, alpha-synuclein protein, and aggregation proteins in neurodegenerative diseases and inflammation in many diseases such as neurodegenerative diseases, cancer and other inflammatory diseases, aggressive cancer tissue, brain activation in neurodegenerative diseases and other diseases by binding and accumulation of the these novel contrast agents to these proteins and diseases by way of the PET machine and/or CT machine and/or SPECT machine; the images contrast (brightness of images) increase as the binding of these agents to the target diseases are increased.

27. The novel contrast agents and imaging methods for early detection of diseases claim 1, comprising: detection of blood brain barrier leakage or break down by using the novel contrast agents such as labeling agent like (morphine, ascorbic acid (vitamin C), carbidopa, dopamine, betaine, congo red, nipecotic acid, guvacine and large polyphenols, and other compounds which can not cross BBB easily) by labelling these agents with the methods as described above in (i) for CT and/or in (iii) for PET to detect blood brain barrier leakage or break down in neurodegenerative diseases, cancer and other diseases by crossing these agents the blood brain barrier at time limit of imaging, such as after 15 mins, 30 mins, 60 mins, or more of administration of the labelled agents by way of the CT machine and/or PET machine; the images contrast (brightness of images) increase as these agents cross blood brain barrier in neurodegenerative diseases and other diseases increase.

28. The novel contrast agents and imaging methods for early detection of diseases claim 1, comprising: detection of aggressive cancer by using the novel contrast agents mentioned in this invention in (i) with CT, and/or in (iii) for PET to detect aggressive cancer tissue by binding and accumulation of these contrast agent to the target proteins of aggressive cancer proteins by way of the CT machine; the CT images and/or PET images contrast (brightness of images) increase as the binding of these agents to the target aggressive cancer tissue increase.

29. The novel contrast agents and imaging methods for early detection of diseases of claim 1, comprising: detection of inflammation in tissue by using the novel contrast agents mentioned in this invention in (i) with CT to detect inflammation in tissue in neurodegenerative diseases and other diseases by binding and accumulation of these contrast agent to the target proteins of inflammation in tissue by way of the CT machine; the CT images contrast (brightness of images) increase as the binding of these agents to the target inflammation in tissue in the inflammatory diseases such as diseases of the skin, diseases of the nervous system, immune diseases, diseases of the musculoskeletal system, concussion, traumatic brain injury, ischemic stroke, cancer, Alzheimer’s disease, Parkinson disease, Huntington’s disease, epilepsy, Down syndrome, Amyotrophic Lateral Sclerosis, Multiple sclerosis and other neurodegenerative diseases and other inflammatory diseases.

30. The novel contrast agents and imaging methods for early detection of diseases of claim 2, comprising: detection of inflammation in tissue by using the novel contrast agents mentioned in this invention in (ii) with SPECT or in (iii) with PET to detect inflammation in tissue in neurodegenerative diseases and other diseases by binding and accumulation of these contrast agent to the target proteins of inflammation in tissue by way of the SPECT machine or the PET machine; the SPECT images contrast or the PET images contrast (brightness of images) increase as the binding of these agents to the target inflammation in tissue in the inflammatory diseases such as diseases of the skin, diseases of the nervous system, immune diseases, diseases of the musculoskeletal system, concussion, traumatic brain injury, ischemic stroke, cancer, Alzheimer’s disease, Parkinson disease, Huntington’s disease, epilepsy, Down syndrome, Amyotrophic Lateral Sclerosis, Multiple sclerosis and other neurodegenerative diseases and other inflammatory diseases.

31. The novel contrast agents and imaging methods for early detection of diseases of claim 1, comprising: detection of brain activation by using the novel contrast agents mentioned in this invention in (i) with CT to detect brain activation in neurodegenerative diseases and other diseases by binding and accumulation of these contrast agent to the target proteins of brain activation by way of the CT machine; the CT images contrast (brightness of images) increase as the binding of these agents to the target brain activation neurodegenerative diseases, cancer and other diseases increase.

32. The novel contrast agents and imaging methods for early detection of diseases of claim 2, comprising: detection of brain activation by using the novel contrast agents mentioned in this invention in (ii) with SPECT or in (iii) with PET to detect brain activation in neurodegenerative diseases and other diseases by binding and accumulation of these contrast agent to the target proteins of brain activation by way of the SPECT or PET machine ; the SPECT or PET images contrast (brightness of images) increase as the binding of these agents to the target brain activation neurodegenerative diseases, cancer and other diseases increase.

33. The novel contrast agents and imaging methods for early detection of diseases of claim 1, comprising: detection of hypoxia by using the novel contrast agents with labelling as mentioned in this invention such as labeling agent like Metronidazole, Tolcapone, Nitroxoline, Chloramphenicol, Oxamniquine, Secnidazole and other drugs containing NO2 and have ability to donate O2, by labelling the compounds as described above in (i) for CT to detect hypoxia in tissue by binding and accumulation of these labeled contrast agent to the target proteins of hypoxia by way of the CT machine; the CT images contrast (brightness of images) increase as the binding of these agents to the target hypoxia in tissue increase.

34. The novel contrast agents and imaging methods for early detection of diseases of claim 2, comprising: detection of hypoxia by using the novel contrast agents with labelling as mentioned in this invention such as labeling agent like Metronidazole, Tolcapone, Nitroxoline, Chloramphenicol, Oxamniquine, Secnidazole and other drugs containing NO2 and have ability to donate O2, by labelling the compounds as described above in (ii) for SPECT or in (iii) for PET to detect hypoxia in tissue by binding and accumulation of these labeled contrast agent to the target proteins of hypoxia by way of the SPECT or PET machine; the SPECT or PET images contrast (brightness of images) increase as the binding of these agents to the target hypoxia in tissue increase.
